(19) United States
(12) Patent Application Publication
(10) Pub. No.: US 2012/0277828 A1
(43) Pub. Date: Nov. 1, 2012

(54) METHODS, DEVICES AND SYSTEMS FOR EFFICIENTLY PROGRAMMING NEUROSTIMULATION

(75) Inventors: Richard W. O'Connor, Redwood City, CA (US); April C. Pixley, Los Altos, CA (US); Daniel M. Brounstein, San Francisco, CA (US); David C. Blaha, Sunnyvale, CA (US)

(73) Assignee: SPINAL MODULATION, INC., Menlo Park, CA (US)

(21) Appl. No.: 13/458,898

(22) Filed: Apr. 27, 2012

Related U.S. Application Data
(60) Provisional application No. 61/481,086, filed on Apr. 29, 2011.

(51) Int. Cl. A61N 1/36

(52) U.S. Cl. 607/59

(57) ABSTRACT

Embodiments of the present invention generally relate to neurostimulation systems, methods for use with neurostimulation systems, and devices (e.g., programmers) of neurostimulation systems. Such a neurostimulation system can include, e.g., a neurostimulator, a programmer configured to communicate with and program the neurostimulator, and one or more leads connected to the neurostimulator, wherein each lead includes one or more electrodes. A method, according to an embodiment of the present invention, is for enabling efficient identification of one or more preferred sets of neurostimulation parameters from among numerous possible sets of neurostimulation parameters, wherein each set of neurostimulation parameters specifies a lead, an electrode configuration for the specified lead, and one or more pulse parameters (e.g., a pulse amplitude value, a pulse width value and/or a pulse frequency value).

### PRIORITY CLAIM

This application claims priority under 35 U.S.C. 119(e) to U.S. Provisional Patent Application No. 61/481,086, filed Apr. 29, 2011, (Attorney Docket No. SPMOD-01005US0), which is incorporated herein by reference.

### FIELD OF THE INVENTION

Embodiments of the present invention relate to methods, devices and systems that are used to provide neurostimulation to treat pain.

### BACKGROUND OF THE INVENTION

Neurostimulation has become an accepted treatment for patients with chronic pain in their back and/or limbs who have not found pain relief from other treatments. In general, neurostimulation comprises applying an electrical current to nerve tissue in the pathway of the chronic pain. This creates a sensation that blocks the brain's ability to sense the previously perceived pain. There are two conventional forms of electrical stimulation commonly used to treat chronic pain: Spinal Cord Stimulation (SCS) and Peripheral Nerve Field Stimulation (PNFS). In SCS, electrical leads are placed along the spinal cord. A programmable implantable neurostimulator (INS) is implanted in the upper buttock or abdomen (under the skin) which emits electrical currents to the spinal cord via electrodes of the leads. PNFS is similar to SCS, however PNFS involves placing the leads just under the skin in an area near to the peripheral nerves involved in pain.

In both approaches, the INS, also known as an implantable pulse generator (IPG), can be programmed with the use of an external programmer. Such programming typically requires the interaction of two or more individuals, e.g., one or more programming person(s) and the patient. A programming person is typically a company representative of the INS manufacturer or a member of a clinical staff. For a specific example of a programming protocol, a programming person may manually adjust the various neurostimulation parameters with the use of the external programmer. The patient gives feedback to the programming person as the programming person adjusts the parameters. The programming person performing the manual adjustments, or a third person, can write down information regarding various stimulus parameters tested and results achieved using the parameters. Thereafter, to retest and/or program previously tested neurostimulation parameters, a programming person manually reenters the previously tested neurostimulation parameters. This is an inefficient and time consuming use of personnel.

Thus, it is desired to provide devices, systems and methods which allow more efficient programming of a neurostimulator for a given patient while reducing and preferably minimizing time and cost. Such devices, systems and methods should improve and preferably optimize the programming parameters while more fully utilizing the capabilities of the neurostimulator and programmer. At least some of these objectives will be met by embodiments of the present invention.

### SUMMARY OF THE INVENTION

Embodiments of the present invention generally relate to neurostimulation systems, methods for use with neurostimulation systems, and devices (e.g., programmers) of neurostimulation systems. Such a neurostimulation system can include, e.g., a neurostimulator (NS), a programmer configured to communicate with and program the NS, and one or more leads connected to the NS, wherein each lead includes one or more electrodes. The NS can be an implantable neurostimulator (INS), or a non-implantable (or at least partially non-implantable) NS, such as a trial neurostimulator (TNS), but is not limited thereto.

A method, according to an embodiment of the present invention, is for enabling efficient identification of one or more preferred sets of neurostimulation parameters from among numerous possible sets of neurostimulation parameters, wherein each set of neurostimulation parameters specifies a lead, an electrode configuration for the specified lead, and one or more pulse parameters (e.g., a pulse amplitude value, a pulse width value and/or a pulse frequency value). The method includes sending instructions to the NS to cause testing each of a plurality of different sets of neurostimulation parameters. A user can manually specify, via the programmer, the lead, the electrode configuration and the one or more pulse parameters corresponding to each of the sets of neurostimulation parameters to be tested. Alternatively, the programmer can (at least partially) automatically select one or more of the lead, the electrode configuration and the one or more pulse parameters corresponding to each of the sets of neurostimulation parameters to be tested. Other variations are also possible.

For each of a plurality of times that an indication is accepted from a user, via the programmer, that information corresponding to a set of neurostimulation parameters being tested should be saved, information (corresponding to the set of neurostimulation parameters being tested) is saved within the programmer and/or the NS. Such an indication can be accepted from a user, e.g., when a user presses a predetermined button (e.g., a “program”, “log” or “save” button) on the programmer. Each instance of the saved information can be referred to as “a snapshot”, and each instance of causing the saving of such information can be referred to as “taking a snapshot”. The tested sets for which information is saved and available for later retrieval can be referred to as a sub-plurality of sets of neurostimulation parameters.

A list indicative of at least some of the tested sets of neurostimulation parameters (for which a user indicated information should be saved) is displayed on the programmer. For example, one list can identify all of the snapshots. Alternatively, where there are a plurality of leads, there can be separate list of snapshots for each of the leads.

The programmer accepts, from a user, a selection of one of the tested sets of neurostimulation parameters from the displayed list. In response one of the tested sets of neurostimulation parameters being selected from the displayed list, instructions are sent to the NS to cause the set of neurostimulation parameters (selected from the displayed list) to be retested without requiring that a user specify, via the programmer, the lead, the electrode configuration and the one or more pulse parameters corresponding to the selected set.

In accordance with an embodiment, while the set of neurostimulation parameters selected from the displayed list is being retested, the programmer can accept, from a user, one or more adjustments to one or more of the pulse parameters of the set of neurostimulation parameters selected from the displayed list.

The programmer can also accept, from a user, a name, rating and/or description of each set of neurostimulation parameters for which information is saved. Thereafter, the list of at least some of the tested sets of neurostimulation parameters (for which the user indicated information should be saved) can be displayed by displaying the corresponding names, ratings and/or descriptions accepted by the programmer. Where the programmer accepts a rating of each set of neurostimulation parameters for which information is saved, the displayed list can be ordered in dependence on the ratings.

In the above summarized method, the programmer can cause information (corresponding to the set of neurostimulation parameters being tested) to be saved in response to accepting a user selection of predetermined button on the programmer. In a specific embodiment, the saved information can be binned based on how many times the predetermined button is selected by a user to indicate that information (corresponding to the set of neurostimulation parameters being tested) should be saved. In such an embodiment, when the list is displayed the list can be ordered in dependence on results of the binning. For example, sets of neurostimulation parameters binned in the same manner can be grouped together in a displayed list

In accordance with an embodiment, one or more groups can be generated in response to a user, via the programmer, grouping together two or more of the tested sets of neurostimulation parameters for which the information was saved. The programmer can then display a list of the one or more groups. Additionally, the programmer can accept, from a user, a selection of one of the displayed groups, in response to which neurostimulation is delivered in accordance with all of the sets of neurostimulation parameters included within the selected group.

In an embodiment, the programmer can accept, from a user, an adjustment to one or more pulse parameters associated with one of the tested sets of neurostimulation parameters for which the information was saved. In response the adjustment to one or more pulse parameters (associated with one of the tested sets of neurostimulation parameters for which the information was saved), there is a modification of the corresponding information saved so that whenever the adjusted set of neurostimulation parameters is again selected, or a group that includes that adjusted set of neurostimulation parameters is again selected, neurostimulation is delivered in accordance with the adjusted one or more pulse parameters.

This summary is not intended to summarize all of the embodiments of the present invention. Further and alternative embodiments, and the features, aspects, and advantages of the embodiments of invention will become more apparent from the detailed description set forth below, the drawings and the claims.

### BRIEF DESCRIPTION OF THE DRAWINGS

FIG. 1A illustrates an exemplary neurostimulation system with which embodiments of the present invention can be implemented.

FIG. 1B is a simplified block diagram that illustrates possible components of the electronic circuitry of the NS shown in FIG. 1A.

FIG. 1C is a simplified block diagram that illustrates possible components of the clinical programmer shown in FIG. 1A.

FIG. 1D is a simplified block diagram that illustrates possible components of the patient programmer shown in FIG. 1A.

FIG. 2 illustrates a portion of an exemplary lead having four electrodes.

FIG. 3 illustrates exemplary programmable pulse parameters of a neurostimulation signal.

FIGS. 4A and 4B show screenshots from a clinical programmer, according to specific embodiments of the present invention.

FIG. 5 is a high level flow diagram used to explain methods according to specific embodiments of the present invention.

FIGS. 6A and 6B show exemplary screen shots of lists of sets of neurostimulation parameters that can be displayed on a clinical programmer, in accordance with specific embodiments of the present invention.

### DETAILED DESCRIPTION OF THE INVENTION

Embodiments of the present invention generally relate to devices, systems and methods that enable efficient programming of a NS by a clinician, physician, patient and/or other programming person (any of which can be referred to more generically as a “user”). The NS can be an implantable neurostimulator (INS), which is also known as an implantable pulse generator (IPG). Before an INS is implanted within a patient to deliver a therapy, a non-implanted NS device that replicates some or all of the INS functions can be connected to the patient to evaluate the efficacy of the proposed therapy. Such a non-implanted NS device is often referred to as a trial neurostimulator (TNS) device. The TNS device can be taped to a patient's back, hooked on a patient's belt, or attached to the patient in some other manner. It is also possible that a non-implanted NS device, similar to a TNS, can be used for extended periods of time, in which case the non-implanted NS device may no longer qualify as a “trial” device. When using such a TNS or other non-implanted NS, stimulation lead(s) that extend from a non-implanted housing or header of the device can be inserted into the patient (e.g., percutaneously) so that distal portions of the lead(s) are positioned at appropriate locations, e.g., along the spinal cord. The same external programmers of embodiments of the present invention (which are described herein as communicating with an INS) can also be used to communicate wirelessly with such a TNS or other non-implanted (or at least partially non-implanted) NS. In other words, the programmers and methods of embodiments of the present invention are not limited to use with an INS.

### Exemplary Neurostimulation System

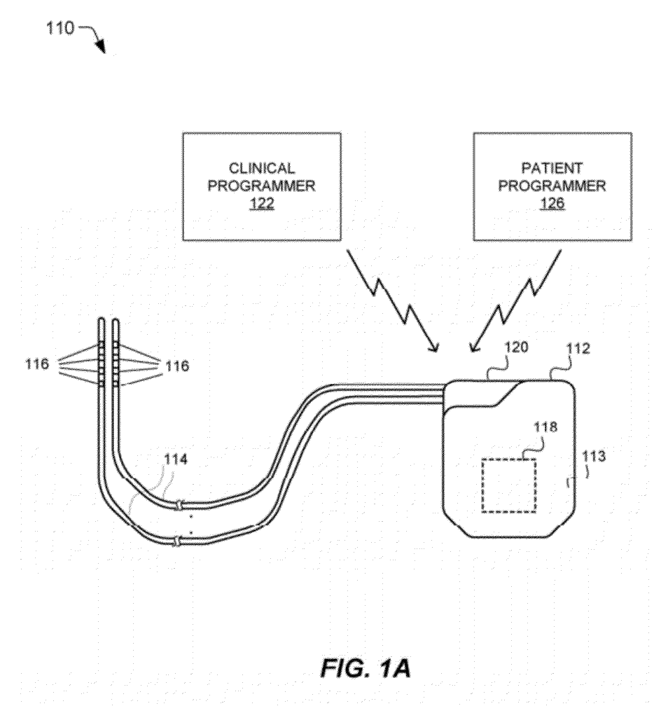

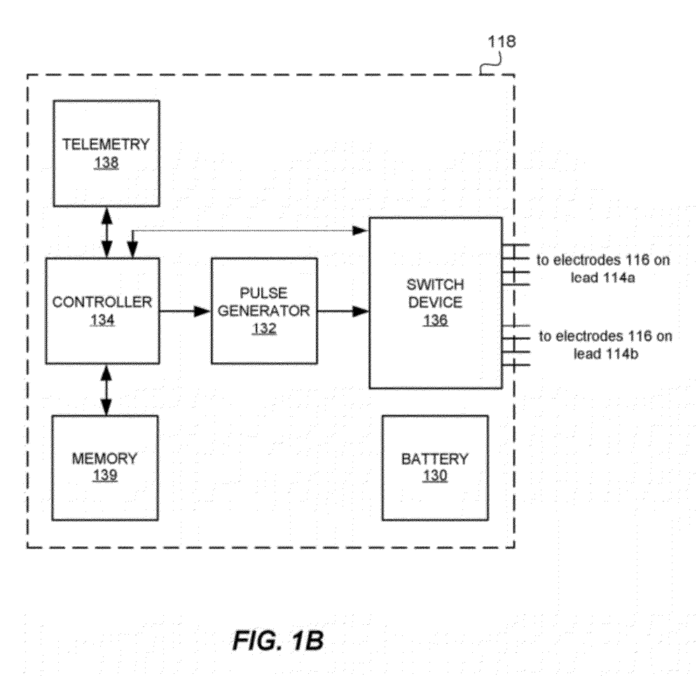

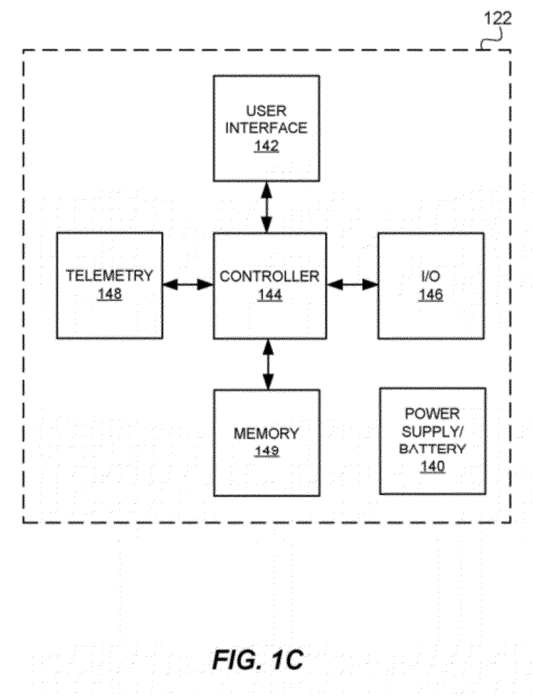

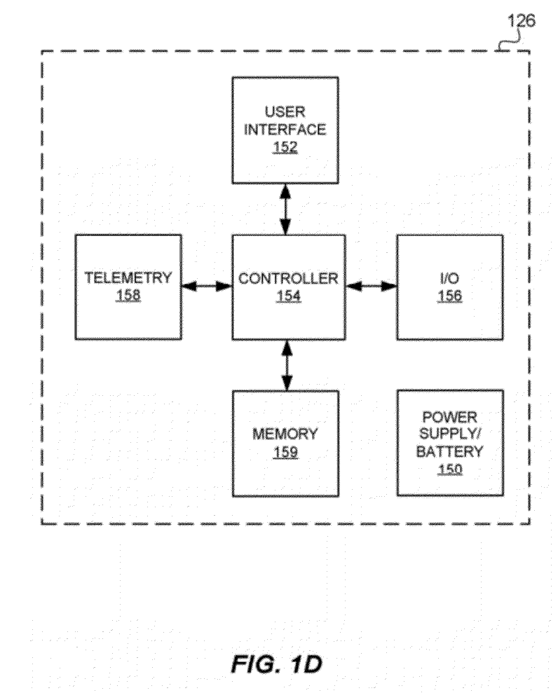

An example neurostimulation system 110 is illustrated in FIG. 1A. In this embodiment, the system 110 includes an NS 112, which can be an INS (typically implantable in a subcutaneous pocket within a patient's body) or an at least partially non-implantable NS (e.g., a TNS), but is not limited thereto. One or more leads 114 are connected to the NS 112, with each lead including one or more electrodes 116. For example, four leads 114 can be connected to the NS 112, with each lead including four electrodes 116. Alternatively, more or less leads can be used, with more or less electrodes per lead. The NS 112 includes electronic circuitry 118 contained within a housing 113 (also referred to as the “case” or “can”) of the INS. The electrodes 116 are electrically coupled to the electronic circuitry 118 by coupling the leads 114 to a connector 120 (also known as a header) of the NS 112. Although not limited thereto, one or more leads 114 can be positioned so that the electrodes are touching, near or within a dorsal root ganglion (DRG). Additional details regarding such neurostimulation are provided in U.S. Pat. No. 7,450,993, entitled “Methods for Selective Stimulation of a Ganglion”, and U.S. patent application Ser. No. 12/607,009, entitled “Selective Stimulation Systems and Signal Parameters for Medical Conditions”, both of which are incorporated herein by reference.

The electronic circuitry 118 can be used to generate and provide an electrically stimulating signal (also referred to as a neurostimulation signal, a neurostimulation waveform, or simply a stimulation signal) to a nerve tissue via at least two of the electrodes 116, with at least one of the electrodes connected as an anode, and at least one of the electrodes connected as a cathode. As described in more detail below, the electronic circuitry can comprise and/or be included within a controller (e.g., processor) for controlling the operations of the device, including stimulating, signal transmission, charging and/or using energy from a battery for powering the various components of the circuitry, and the like.

A programmer 122 and/or 126 can be used to program various neurostimulation parameters and/or other instructions into the electronic circuitry 118. The programmer can include a processor (or other type of controller) and memory that can store one or more code modules. The processor or other controller can execute one or more code modules to perform programming of the NS 112 based on a programming protocol, input from a programming person and feedback from a patient. For example, the programmer 122 or 126 can transmit instructions to the NS 112 that instruct the NS to test specific neurostimulation parameters, adjust certain parameters, and/or program certain parameters as those to be used by the NS for chronic treatment of pain. The programmer 122 or 126 may include (or be coupled to) telemetry circuitry that communicates with the electronic circuitry 118 via radio frequency (RF) or other wireless signals. Regardless whether the telemetry circuitry is within a programmer or coupled to the programmer, the programmer can be said to include telemetry circuitry. The NS 112 can similarly include a processor (or other type of controller) that can execute code modules stored in memory of the NS.

There are generally two types of programmers, both of which can be handheld and capable of wireless communication with the NS 112. The programmer represented by block 122, which is often referred to as a “clinical programmer” (or sometimes referred to as a “clinician programmer”), may be used by a representative of the NS manufacturer, a clinician, a physician and/or other medical personnel (any of which can be referred to hereafter as a “programming person” or as a “user”). Block 126 represents another type of programmer, which is often referred to as a “patient programmer,” which is primarily intended to be controlled by the patient being treated by the NS 112.

FIG. 1B is a simplified block diagram that illustrates possible components of the electronic circuitry 118 of the NS 112 shown in FIG. 1A. Referring to FIG. 1B, the electronic circuitry 118 is shown as including a battery 130, a pulse generator 132, a controller 134, a switch device 136, telemetry circuitry 138 and memory 139.

The battery 130 can be used to power the various other components of the electronic circuitry 118. Further, the battery 130 can be used to generate stimulation pulses. As such, the battery can be coupled to the pulse generator 132, the controller 134, the switch device 136, the telemetry circuitry 138 and the memory 139. One or more voltage regulators (not shown) can step up or step down a voltage provide by the battery 130 to produce one or more predetermined voltages useful for powering such components of the electronic circuitry 118.

The pulse generator 132 can be coupled to the electrodes 116 of the lead(s) 114 via the switch device 136. The pulse generator 132 can include circuitry, such as capacitors, resistors, transistors, and the like, which are used to generate stimulation pulses, as is well known in the art. The pulse generator 132 can be a single- or multi-channel pulse generator, and can be capable of delivering a single stimulation pulse or multiple stimulation pulses at a given time via a single electrode combination or multiple stimulation pulses at a given time via multiple electrode combinations.

The controller 134 can control the pulse generator 132 to generate stimulation pulses, and control the switch device 136 to couple the stimulation energy to selected electrodes 116 of one or more selected leads 114. Additionally, the controller 134 can control the switch device 136 to select different electrode configurations for delivery of stimulation energy from the pulse generator 132. More specifically, the controller 134 can control the pulse generator 132 and the switch device 136 to deliver stimulation energy in accordance with parameters specified by one or more sets of neurostimulation parameters, which may be stored within the memory 139. Each set of neurostimulation parameters can specify a lead, an electrode configuration for the specified lead, and one or more pulse parameters. For example, where there are four leads, a set of neurostimulation parameters can specify which of the four leads is selected. Where each lead includes four electrodes, a set of neurostimulation parameters can specify how each of the four electrodes of a selected lead is configured, e.g., as an anode (having a positive polarity), a cathode (having a negative polarity), or as an inactive electrode (in which case the electrode is not used for delivering stimulation energy). Exemplary programmable pulse parameters that can be specified include pulse amplitude, pulse width, and pulse frequency (also known as pulse repetition rate) for a stimulation signal.

The controller 134 can include a microprocessor, a microcontroller, a digital signal processor (DSP), an application specific integrated circuit (ASIC), a field-programmable gate array (FPGA), a state machine, or similar discrete and/or integrated logic circuitry. The switch device 136 can include a switch array, switch matrix, multiplexer, and/or any other type of switching device suitable to selectively couple stimulation energy to selected electrodes. The memory 139 can include RAM, ROM, NVRAM, EEPROM or flash memory, but is not limited thereto. Various sets of neurostimulation parameters can be stored in the memory 139, examples of which are discussed herein.

FIG. 1C is a simplified block diagram that illustrates possible components of the clinical programmer 122 shown in FIG. 1A. Referring to FIG. 1C, the clinical programmer 122 is shown as including a power supply 140, a user interface 142, a controller 144, input and output (I/O) circuitry 146, telemetry circuitry 148 and memory 149.

The power supply 140, which can include a battery, can be used to power the various other components of the clinical programmer 122. As such, the power supply 140 can be coupled to the user interface 142, the controller 144, the I/O circuitry 146, the telemetry circuitry 148 and the memory 149. One or more voltage regulators (not shown) can step up or step down a voltage provided by a battery or an external power source to produce one or more predetermined voltages useful for powering such components of the clinical programmer 122.

A programming person may interact with the controller 144 via the user interface 142 in order to test various sets of neurostimulation parameters, input user feedback, select preferred or optimal neurostimulation parameters, and the like. The user interface 142 can include a display, a keypad, a touch screen, mechanical buttons, one or more peripheral pointing devices (e.g., a mouse, touchpad, joystick, trackball, etc.), and/or the like. The controller 144 can provide a graphical user interface (GUI) via the user interface 142 to facilitate interaction with a clinician or physician. Alternative types of user interfaces, e.g., one they relies primarily on mechanical type button, knows, switches, etc. may also be used. It is also possible that the programmer 122 include voice recognition capabilities, so that feedback received from the patient and/or programming person can be accepted verbally by the programmer 122. The controller 144 can include a microprocessor, a microcontroller, a digital signal processor (DSP), an application specific integrated circuit (ASIC), a field-programmable gate array (FPGA), a state machine, or similar discrete and/or integrated logic circuitry. The I/O circuitry 146 can include one or more transceivers for wireless communication, ports for wired communication and/or communication via removable electrical media, and/or appropriate drives for communication via removable magnetic or optical media. The telemetry circuitry 148 can include an RF transceiver that is connected to an antenna.

As described in addition detail below, the controller 144 can collect information relating to tested sets of neurostimulation parameters, and store the information in the memory 149 for later retrieval and review by a clinician, physician or by the controller 144 to facilitate identification of one or more preferred sets of neurostimulation parameters. The controller 144 can send instructions to the NS 112 via the telemetry circuit 148 to cause the testing of various sets of neurostimulation parameters. For example, the controller 144 can effectuate testing, by the NS 112, of various sets of neurostimulation parameters created by the controller 144 or a programming person.

The memory 149 can include program instructions that, when executed by the controller 144, cause the programmer 122 to perform at least some of the functions described herein. For example, the controller 144 can execute program instructions that specify protocols for testing various sets of neurostimulation parameters and selecting one or more preferred sets of neurostimulation parameters. The memory 149 can also store one or more sets of neurostimulation parameters determined to treat targeted pain for a patient, along with information about the patient. The memory 149 can include any volatile, non-volatile, fixed, removable, magnetic, optical, or electrical media, such as a RAM, ROM, CD-ROM, hard disk, removable magnetic disk, memory cards or sticks, NVRAM, EEPROM, flash memory, and the like.

FIG. 1D is a simplified block diagram that illustrates possible components of the patient programmer 126 shown in FIG. 1A. Referring to FIG. 1D, the patient programmer 126 is shown as including a power supply 150, a user interface 152, a controller 154, input and output (I/O) circuitry 156, telemetry circuitry 158 and memory 159. The power supply 150, which can include a battery, can be used to power the various other components of the patient programmer 126. As such, the power supply 150 can be coupled to the user interface 152, the controller 154, the I/O circuitry 156, the telemetry circuitry 158 and the memory 159. One or more voltage regulators (not shown) can step up or step down a voltage provide by a battery or an external power source to produce one or more predetermined voltages useful for powering such components of the patient programmer 126.

A patient can interact with the controller 154 via the user interface 152 in order to select, modify or otherwise control delivery of neurostimulation therapy. For example, the patient may be able to select among various sets of neurostimulation parameters that are stored in the memory 159. Additionally, or alternatively, the patient may be able to increase or decrease specific neurostimulation signal parameters, such as pulse amplitude, to tailor the therapy to the pain being experienced at the time. The user interface 152 can additionally, or alternatively, provide various other functions. The user interface 152 can include a display, a keypad, a touch screen, mechanical buttons, one or more peripheral pointing devices (e.g., a mouse, touchpad, joystick, trackball, etc.), and/or the like. The controller 154 can provide a graphical user interface (GUI) via the user interface 152 to facilitate interaction with a patient. The user interface 152 of the patient programmer 126 often, but not necessarily, provides less capabilities than the user interface 142 of the clinical programmer 122. The controller 154 and the I/O circuitry 156 of the patient programmer can be similar to, but will likely include less capabilities than, the controller 144 and the I/O circuitry 146 of the clinical programmer 122.

In some embodiments, the memory 159 can store data related to sets of neurostimulation parameters that are available to be selected by the patient for delivery of neurostimulation therapy to the patient being treated by the NS 112. In some embodiments, the controller 154 can record usage information and store usage information in the memory 159. The memory 159 can include program instructions that, when executed by the controller 154, cause the patient programmer 126 to perform functions ascribed to the patient programmer 126. The memory 159 can include any volatile, non-volatile, fixed, removable, magnetic, optical, or electrical media, such as a RAM, ROM, CD-ROM, hard disk, removable magnetic disk, memory cards or sticks, NVRAM, EEPROM, flash memory, and the like.

The telemetry circuitry 158 allows the controller to communicate with NS 112, and the I/O circuitry 156 allows the controller 154 to communicate with the clinical programmer 122. The controller 154 can receive selections of, or adjustments to, sets of neurostimulation parameters made by the patient via the user interface 152, and can transmit the selection or adjustment to the NS 112 via telemetry circuitry 158. Where the patient programmer 126 stores data relating to sets of neurostimulation parameters in the memory 159, the controller 154 can receive such data from the clinical programmer 122 via the I/O circuitry 156 during programming by a clinician or physician. Alternatively, the patient programmer 126 can use the telemetry circuitry to upload, from the NS 112, sets of neurostimulation parameters that have been programmed into the NS 112 by the clinical programmer 122. The telemetry circuitry 158 can include an RF transceiver that is connected to an antenna.

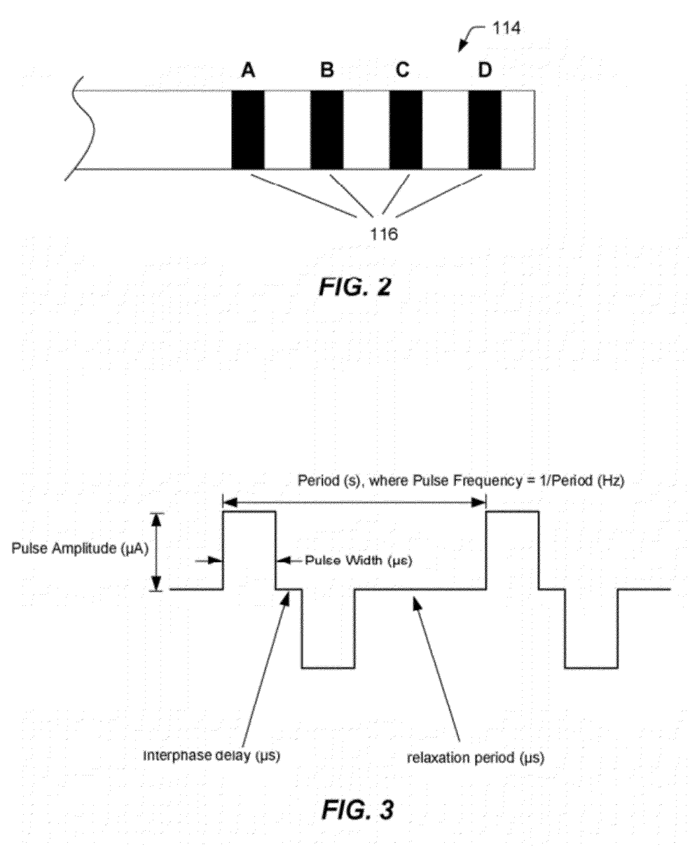

FIG. 2 illustrates a portion of an exemplary lead 114 having four electrodes 116 (labeled A, B, C, D). Such electrodes 116 can be placed, e.g., within, touching or in close proximity to the target nerve tissue, such as the DRG, but is not limited thereto. When a given electrode is selected to receive an electrical stimulus, it is (for purposes of the present invention) said to be “activated” or used to deliver neurostimulation. When an electrode is not selected to receive an electrical stimulus, it is said to be “non-activated,” “inactive,” “neutral,” or not used to deliver neurostimulation. Electrical neurostimulation occurs between two or more electrodes so that the electrical current associated with the stimulus has a path from the NS 112 to the tissue to be stimulated, and a return path from the tissue to the NS 112. Bipolar stimulation occurs when two of the electrodes of the leads 116 are activated, e.g., when one electrode 116 is activated as an anode at the same time that another electrode 116 is activated as a cathode. Tripolar stimulation occurs when three of the electrodes 116 of the lead(s) 114 are activated, e.g., two electrodes 116 can be activated as an anode at the same time that another electrode 116 is activated as a cathode. In general, multipolar stimulation occurs when multiple electrodes 116 of the lead(s) 114 are activated.

### Exemplary Neurostimulation Signal

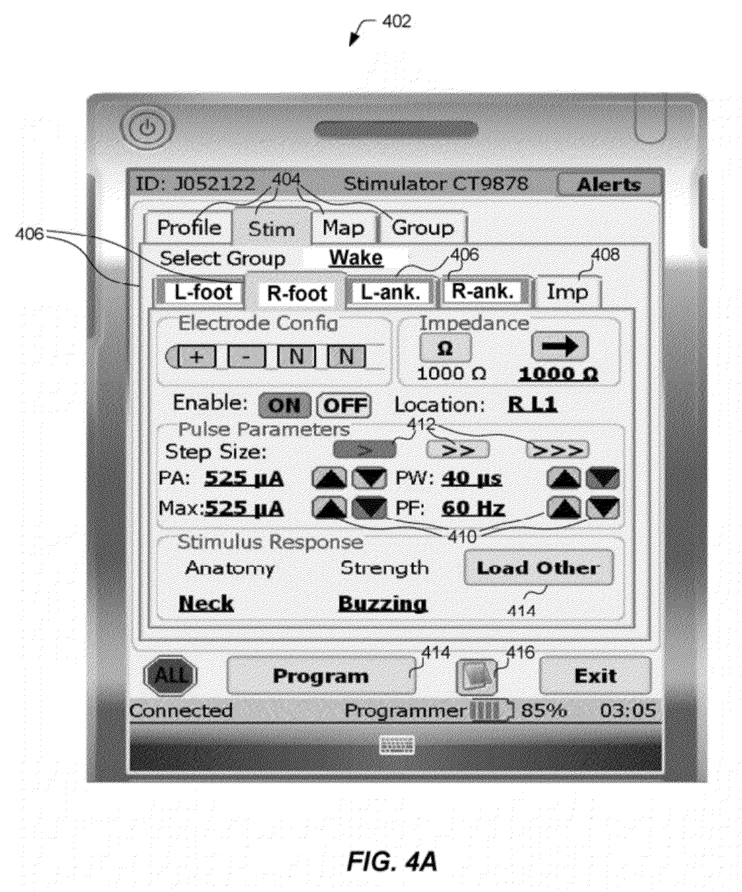

FIG. 3 is used to illustrate exemplary pulse parameters of an exemplary neurostimulation signal that includes biphasic pulses. The pulse parameters include pulse amplitude, pulse width and pulse frequency. In FIG. 3 the pulse amplitude is shown as specifying a baseline-to-peak amplitude, but can alternatively define a peak-to-peak amplitude. The pulse amplitude can be specified, e.g., in microamps (μA) and/or millivolts (mV), but is not limited thereto. The pulse width can be specified, e.g., in microseconds (μs), but is not limited thereto. The pulse frequency can be specified, e.g., in hertz (Hz), but is not limited thereto.

Each biphasic pulse is shown as including a positive portion and a negative portion. In one embodiment, the negative portion of the biphasic stimulation pulse can be achieved by inverting the anode/cathode configuration that was used to achieve the positive portion of the biphasic pulse, but this need not be the case. In FIG. 3, the positive and negative portions of the biphasic pulse are separated by an interphase delay, which can be fixed or can be programmable. It is also possible that there is no interphase delay. Consecutive pulses can be separated by a baseline relaxation period, which charge residual on electrodes used to deliver the stimulation pulse may be discharged. FIG. 3 has been included solely to explain an exemplary neurostimulation signal having corresponding exemplary pulse parameters. However, embodiments of the present invention can be used to provide various other types of neurostimulation signals. For example, neurostimulation signals that includes pulses other than biphasic pulses can be used, e.g., monophasic or triphasic pulses can be used, or other types of biphasic pulses can be used. These are just examples, which are not meant to be limiting.

### Sets Of Neurostimulation Parameters

A set of neurostimulation parameters that defines how neurostimulation (also referred to simply as stimulation) is to be delivered using a specific lead can also be referred to as a “lead set” since the set defines the stimulation therapy to be delivered to a patient via a lead. One or more sets of neurostimulation parameters can be grouped together into a group, which can be referred to as a “stimulation set”. One set of neurostimulation parameters (i.e., one “lead set”) may be useful for treating a condition in one location of the body of the patient, while a second set of neurostimulation parameters (i.e., a second “lead set”) may be useful for treating a condition in a second location of the body. If both such “lead sets” are grouped together into the same group (i.e., the same “stimulation set”), then both the first and second locations of the body can be treated when that group is selected. It is also possible that more than one “lead set” grouped into the same “stimulation set” can be used for providing treatment for a common location of the body. It is also possible that one set of neurostimulation parameters provides a first response (e.g., tingling) to a location of the body, while another set of neurostimulation parameters provides a second response (e.g., massaging) to the same location of the body. Other variations are also possible.

In an embodiment, the data for each set of neurostimulation parameters includes lead information, electrode configuration information and pulse parameter information. More specifically, in an embodiment the data for each set of neurostimulation parameters (i.e., for each “lead set”) specifies one of the leads, how each of the multiple electrodes (e.g., four electrodes) of the lead is to be configured (i.e., as an anode electrode, a cathode electrode, or an inactive electrode), and specifies a pulse amplitude, a pulse width and a pulse frequency of the stimulation waveform to be delivered using that lead and electrode configuration. In an embodiment, data for each group (i.e., each “stimulation set”) specifies which one or more sets of neurostimulation parameters (e.g., up to four sets of neurostimulation parameters) are included as part of the group. For example, if there are twelve sets of neurostimulation parameters (i.e., lead set 1, lead set 2, . . . lead set 12), an exemplary group can include lead set 2, lead set 5, lead set 6 and lead set 11. During a period of time that a specific group is selected/active, stimulation can be delivered using all four leads, e.g., in a time multiplexed manner (although, at any moment in time during that period it may be that stimulation can only be delivered using one of the leads). More than one set of neurostimulation parameters can specify the same lead (i.e., can have the same lead information), yet have different electrode configuration information and/or different pulse parameter information. For example, lead set 1, lead set 2, and lead set 9 can each specify different ways that stimulation can be delivered using the same lead. However, in some embodiments, two lead sets specifying the same lead can not be grouped into the same group (i.e., into the same “stimulation set”).

### Exemplary Screen Shots

As discussed above, the clinical programmer 122 includes a user interface 142 that enables a programming person (also referred to as a “user”) to interface with the programmer 122. In accordance with an embodiment, the user interface 142 is a touch screen type of graphical user interface (GUI) that includes multiple screens that can be displayed to the programming person. Such a GUI can be controlled by the controller 134 of the programmer 122.

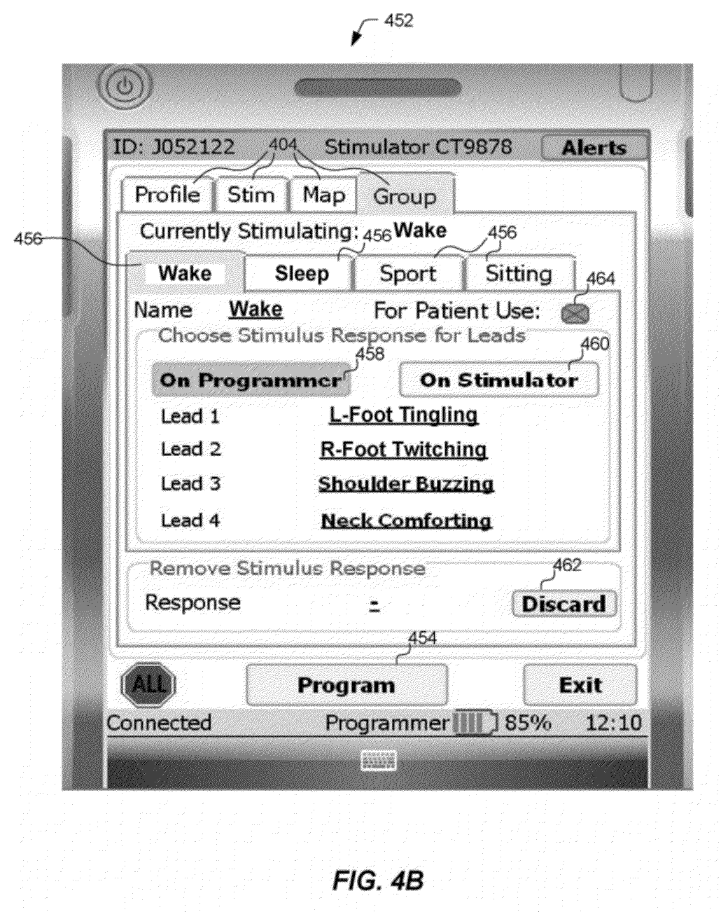

FIG. 4A shows an example screenshot 402 of the user interface 142 of the clinical programmer 122. Referring to FIG. 4A, the user interface 142 of the clinical programmer 122 is shown as including “workspaces” that are used to view and adjust the neurostimulation parameter settings and to obtain diagnostic information. In some embodiments, four workspaces are provided, including: “Profile”, “Stim”, “Map” and “Group”. The four workspaces are shown as workspace tabs 404 near the top of the screenshot 402. Additional and/or alternative workspaces can also be available. Additional details of the “Stim” and “Group” workspaces are discussed below.

The screenshot 402 corresponds to the “Stim” workspace being selected. As can be appreciated from FIG. 4A, the Stim workspace can be used to selectively activate (turn on) a lead; select/adjust electrode configurations; and select/adjust pulse parameters. There can be one lead tab 406 for each lead, wherein each lead tab 406 may be named (also referred to as “labeled”) with the corresponding body region treated by neurostimulation. FIG. 4A illustrates four body region tabs 406, one each for left foot, right foot, left ankle and right ankle.

As mentioned above, in some embodiments each lead has four electrodes. Each of the electrodes can be programmed as an anode (+), a cathode (−) or as inactive, also referred to as neutral (N).

The pulse parameters are also programmable. As mentioned above, the pulse parameters can include: pulse amplitude (PA), pulse width (PW) and pulse frequency (PF). In an embodiment, there can also be a maximum allowable pulse amplitude (Max), which places an upper limit on the pulse amplitude. Each of the pulse parameters can be increased or decreased by pressing the appropriate “Up” or “Down” arrow button 410. In an embodiment, selectable step size buttons 412 can be used when changing pulse parameter settings. For example, the programming person may start with a larger step size (>>>) for gross changes in parameter values and then move to a smaller step size (>>) and even smaller step size (>) when approaching the desired parameter value. The actual step sizes corresponding to the selectable step size buttons 412 can be preprogrammed into the programmer 122. As the programming person scrolls through different ranges of the parameter values and/or different pulse parameters, the step size can automatically change to a granularity appropriate for the range and/or pulse parameter.

FIG. 4B shows a further exemplary screenshot 452 of the user interface 142 of the clinical programmer 122. The screenshot 452 corresponds to the “Group” workspace being selected, via selection of the Group one of the tabs 404. As can be appreciated from FIG. 4B, the Group workspace can be used to selectively group multiple sets of stimulation parameters (i.e., multiple lead sets) into a group (i.e., into a stimulation set). Four group tabs 456 are shown in the screenshot 452, which enables a user to select four different group screens. The names on the tabs 456 correspond to the names of the groups, which in this example, include: “Wake”, “Sleep”, “Sport”, and “Sitting”. In certain embodiments (where two sets of neurostimulation parameters specifying the same lead can not be grouped into the group), up to four sets of neurostimulation parameters (i.e., one for each of four leads) can be grouped into a same group. In the screenshot 452, the group named “Wake” includes: a set of neurostimulation parameters named “L-Leg Tingling” associated with Lead 1; a set of neurostimulation parameters named “R-Foot Twitching” associated with Lead 2; a set of neurostimulation parameters named “Shoulder Buzzing” associated with Lead 3; and a set of neurostimulation parameters named “Neck Comforting” associated with Lead 4.

An “On Programmer” button 458 can be used to group sets of neurostimulation parameters stored within the memory (e.g., 149) of the clinical programmer 122. An “On Stimulator” button 460 can be used to group sets of neurostimulation parameters stored within the memory (e.g., 139) of the NS 112. A “Program” button 454 on the Group workspace can be used to group the sets of neurostimulation parameters (whose names are currently displayed) together as part of the group whose tab (“Wake” in FIG. 4B) is currently selected and displayed.

A “For Patient Use” button 464 can be used by a programming person to specify whether a patient can select/activate, via a patient programmer 126, certain sets of neurostimulation parameter and/or groups for which information is stored in memory (e.g., 139) of the NS implanted in the patient.

A “Discard” button 462 can be used to delete information corresponding to a set of neurostimulation parameters that was previously stored in memory (e.g., 139) of the NS 112 and/or in memory (e.g., 149) of the programmer 122. This is useful where it has been determined that one of the sets previously considered to be of interest is no longer considered to be of interest, e.g., because a more preferred set was found.

While exemplary screen shots have been shown and described, most if not all of the features provided using such screens can be provided using alternative GUIs, or less graphical or non-graphical user interfaces. Accordingly, embodiments of the present invention described below are not limited to use with a GUI similar to those described with reference to FIGS. 4A and 4B.

### Efficient Identification of Preferred Sets of Neurostimulation Parameters

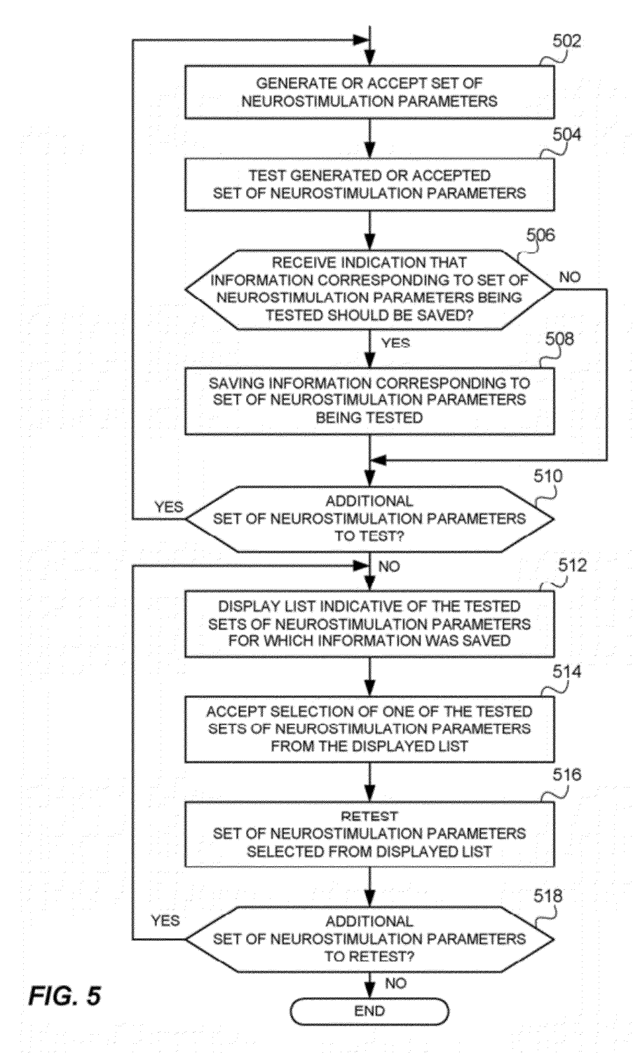

As discussed above with reference to FIG. 1A, a neurostimulation system can include multiple (e.g., four) implantable leads, with each lead including multiple (e.g., four) electrodes. As also mentioned above, a set of neurostimulation parameters can be used to specify a lead, an electrode configuration for the specified lead, and one or more pulse parameters that are used for delivering neurostimulation using the specified lead and electrode configuration. For each lead, one or more electrodes on the lead can be used as a cathode, while one or more electrodes on the lead (or potentially a different lead) can be used as an anode, and one or more electrodes on the lead can be inactive. Accordingly, there are numerous possible electrode configurations for each of the multiple leads. As also mentioned above, the pulse parameters used to specify a neurostimulation signal can include, e.g., pulse amplitude, pulse width and pulse frequency. Depending on a specific implementation, where there are multiple leads there can be an almost indefinite number of different combinations of electrode configurations and pulse parameters for each of the leads. Accordingly, it would not be practical from cost and time perspectives to test and save information for every possible combination of neurostimulation parameters. Rather, what is needed are methods, devices and systems for efficiently identifying a reasonable number of preferred sets of neurostimulation parameters from among the almost indefinite number of possible sets of neurostimulation parameters. Additionally, it would be useful if the various sets of neurostimulation parameters can be readily compared to one another and easily modified if necessary or desired. Examples of such methods, devices and systems, according to embodiments of the present invention, shall be explained with reference to FIG. 5. As will be appreciated from the following description, the flow diagram of FIG. 5 is primarily from the perspective of a programmer device (e.g., a clinical programmer 122).

Referring to FIG. 5, at step 502, a programmer (e.g., 122) generates or accepts a set of neurostimulation parameters that is to be tested. For example, the programmer can automatically generate a set of neurostimulation parameters using an algorithm (e.g., specified by software and/or firmware) and specified ranges of the pulse parameters. Alternatively, a user can manually enter, using a user interface (e.g., 142), information pertaining to the set of neurostimulation parameters to be tested. For another example, numerous sets of neurostimulation parameters that are to be tested can be preprogrammed into the programmer, and the programmer can automatically cycle through the sets. Other variations are also possible. Either way, the programmer (using its telemetry circuitry, e.g., 148) can send instructions to an NS (e.g., NS 112) to cause the testing of the generated or accepted set of neurostimulation parameters. In response to receiving such instructions, the NS can temporarily store (e.g., in volatile memory, such as RAM) data that defines the set of neurostimulation parameters, and the NS tests the set of neurostimulation parameters, as indicated at step 504. Such testing preferably takes place for at least an amount of time (e.g., at least 10 seconds) that is sufficient for the patient (being treated by the NS) to contemplate whether and/or to what extent the set of neurostimulation parameters effectively treats a targeted pain, or causes an undesirable yet potentially informative sensation (e.g., a burning sensation or a muscle twitch).

As part of a testing protocol, the patient can provide feedback (e.g., verbally) to the programming person regarding the set of neurostimulation parameters being tested. Based on that feedback, the programming person can decide whether or not the set of neurostimulation parameters being tested is of sufficient interest that information corresponding to the set of neurostimulation parameters being tested should be saved and thereby be available for later retrieval. The programming person can select (e.g., press) a predetermined button (e.g., a “save”, “program” or “log” button) on the programmer if they decide that the set of neurostimulation parameters being tested is of sufficient interest that information corresponding to the set should be saved. Such a button can be, e.g., a mechanical push-button or a button on a touch screen, but it not limited thereto. It is also possible that the patient can interact directly with the programmer. In other words, the patient may act as the programming person. Further, while interaction with the programmer has usually been described as involving a user pressing or otherwise physically interfacing with the programmer, most if not all of the interactions described herein can be implemented using a voice controlled user interface.

As indicated at steps 506 and 508, in response to accepting an indication from the user (that information corresponding to a set of neurostimulation parameters being tested should be saved), the programmer causes the saving (within the programmer and/or NS) of information corresponding to the set of neurostimulation parameters being tested. In general, whenever the programmer causes the NS to do something (e.g., such as save specific information), the programmer can do so be sending appropriate instructions to the NS. The information saved at step 508 is saved in a manner that enables the saved information to be later retrieved (in contrast to the temporary saving of neurostimulation parameters that may be required to test a set of neurostimulation parameters). For example, at step 508 the information can be saved within a non-volatile portion of memory (e.g., 139) of the NS and/or within a non-volatile portion of memory (e.g., 149) of the programmer 122. A single button (e.g., a “program” button) can be used to save information in both the NS and the programmer. Alternatively, one button (e.g., a “save” button) can cause the saving of the information in the NS, while another button (e.g., a “log” button) can cause the saving of the information in the programmer.

The information saved at step 508 can specify the lead, the electrode configuration for the specified lead, and the one or more pulse parameters of the set of neurostimulation parameters. The information saved at step 508 can be referred to as a “snapshot” since such information is indicative of the neurostimulation being delivered at a particular point in time. Similarly, a user can be said to be “taking a snapshot” when they press the predetermined button (e.g., a “save”, “program” or “log” button) that causes the saving of a snapshot. The tested sets for which information is saved and available for later retrieval can be referred to as a sub-plurality of sets of neurostimulation parameters. Information regarding the date and/or time at which each snapshot is saved can also be saved. A snapshot may be saved because it corresponds to set of neurostimulation parameters deemed to be important by the user. For example, one snapshot can include a set of neurostimulation parameters corresponding to a perception threshold where a patient just starts to perceive paresthesia at a location of the body, while another snapshot can include a set of neurostimulation parameters corresponding to a muscle stimulation threshold where a patient starts to perceive muscle twitching. Still another snapshot can include a set of neurostimulation parameters that the patient indicated provided complete pain relief. These are just a few examples of the types of snapshots that might be saved. Even though the “log” button is an example of the type of button that can be used to save a snapshot, the saved snapshots are not the same as typical session log, because a typical session log would include information about every set of neurostimulation parameters tested, whether or not it was deemed to be important by the user. Thus, if one-hundred different sets of neurostimulation parameters were tested during a programming session, a typical session log would include a separate entry for each of the one-hundred sets tested. By contrast, if the user only deemed that five (of the one-hundred tested sets) were important, the user would only save five snapshots. Accordingly, it should be much more efficient for the user to review a list of snapshots, as compared to reviewing an entire session log. Additionally, it should take less time for a user to compare snapshots, adjusts snapshots, and select which snapshots should be programmed into the NS and/or patient programmer for chronic treatment of pain, as compared to if the user had to select from among all the sets in an entire session log.

In an embodiment, when information corresponding to a set of neurostimulation parameters is saved, an alphanumeric character (e.g., 1, 2, 3 . . . or A, B, C . . . ) can be assigned to the set by the programmer and saved as the name for the set. Alternatively, the programmer can accept, from the programming person, a name, rating and/or description of each set of neurostimulation parameters for which information is saved, in which case the name, rating and/or description can also be saved. In other words, a name, rating and/or description can be saved for each snapshot. The name, rating and/or description can be entered, e.g., using a mechanical or touch key pad, one or more drop-down or pop-up menus, a list box, or the like. Where a key pad is used, freeform text may be entered. Where one or more drop-down or pop-up menus or list boxes is/are used, the name, rating and/or description may be limited to predetermined selections that can be selected from the menu(s) and/or list(s). The name and/or description accepted for a set of neurostimulation parameters can relate to an anatomical region (also referred to as location of the body) treated by the set and/or the response provide by the set (e.g., tingling, buzzing, massaging, etc.), but is not limited thereto.

As indicated by the NO branch of step 506, if the programmer does not receive an indication from the user that information corresponding to a set of neurostimulation parameters being tested should be saved, then such information is not saved and not available for later retrieval. In an embodiment, the programmer can be configured such that the programming person must select another predetermined button (e.g., a “next”, “do not save”, “do not program” or “do not log” button) on the programmer if they decide that the set of neurostimulation parameters being tested is not of sufficient interest that information corresponding to the set should be saved. Alternatively, the programmer can be configured such that if the user does not select at least one predetermined button (e.g., a “save” “program” or “log” button) within a predetermined amount of time (e.g., 30 seconds), the programmer can interpret that as an indication that information corresponding to the set should not be saved. This would be especially useful where the programmer automatically generates the sets of neurostimulation parameters being tested. For example, if the user does not select the predetermined button (e.g., the “program” or “log” button) within a predetermined amount of time (e.g., 30 seconds) after testing of a set of neurostimulation parameters has started, then the programmer can automatically progress to effect testing of a next set of neurostimulation parameters. In an embodiment where the user manually enters information (e.g., lead selections, electrode configurations and pulse parameters) pertaining to the sets of neurostimulation parameters to be tested, if the user manual adjusts a set of neurostimulation parameters while the set is being tested, or enters a completely new set of neurostimulation parameters to be tested, without first selecting at least one predetermined button (e.g., a “save”, “program” or “log” button), then information regarding the previously tested set (i.e., the set prior to modification) is not saved and not available for later retrieval.

As indicated by step 510, the above described steps are repeated so long as there are additional sets of neurostimulation parameters to test. There can be additional sets to test because the programming person wants to test more sets, because the programmer has been programmed to automatically test more sets and/or because a predetermine amount of time allocated to testing sets of neurostimulation parameters has not yet been used up. Regardless, there is preferably a predetermined button (e.g., a “stop all” button) and/or protocol (e.g., selection of another screen on the programmer) that allows the programming person to end the testing at any given time.

As indicated at step 512, the programmer can display a list indicative of (at least some of, or all of) the tested sets of neurostimulation parameters for which the programming person indicated information should be saved. In other words, at step 512 the programmer can display a list of the saved snapshots, or a list of at least some of the saved snapshots. The list can be displayed, e.g., in response to the programming person pressing a predetermined button (e.g., the “load other” button 414 in FIG. 4A, or a “list snapshots” button) on the programmer. In one embodiment, the list that is displayed includes all of the tested sets for which the programming person indicated information should be saved (i.e., the list can include all saved snapshots). If such a list does not all fit on the display screen of the programmer at the same time, then the user can use the user interface of the programmer to scroll down (or up) as necessary.

In one embodiment, there is a separate list associated with each separate lead. For example, if the system includes four leads, and each set of neurostimulation parameters is for use with only one of the four leads, then there can be four separate lists indicative of the tested sets of neurostimulation parameters for which the programming person indicated information should be saved. This embodiment is especially useful where each separate lead is used to treat pain in a separate location of the body, because this embodiment allows the efficacy of different sets of neurostimulation parameters affecting a same location of the body to be readily compared to one another.

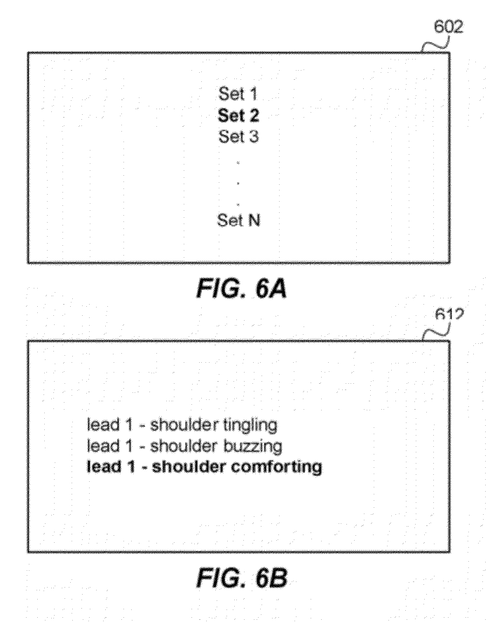

FIG. 6A illustrates one example of a list 602 that can be displayed at step 512. FIG. 6B illustrates another example of a list 612 that can be displayed at step 512. In certain embodiments, explained above, a separate list similar to the list 612 shown in FIG. 6B can be displayed for each separate lead. In an embodiment, where the user can enter a name, rating and/or description of each set of neurostimulation parameters for which information is saved, the names, ratings and/or descriptions can be shown in the list, e.g., as shown in FIG. 6B.

Referring back to FIG. 5, at step 514 the programmer accepts, from a user, a selection of one of the tested sets of neurostimulation parameters from the displayed list. If the list is displayed on a touch screen, one of the sets can be selected by touching or tapping on a name, rating and/or description of one of the sets. Alternatively, a keypad or peripheral pointing devices (e.g., a mouse, touchpad, joystick, trackball, etc.) of the user interface can be used to select one of the sets from the list.

At step 516, in response one of the tested sets of neurostimulation parameters being selected from the displayed list, the selected set of neurostimulation parameters is retested without requiring that a user specify, via the programmer, the lead, the electrode configuration and the one or more pulse parameters corresponding to the selecting one of the tested sets of neurostimulation parameters. More specifically, the programmer sends such instructions to the NS, and the NS performs the retesting. This allows for efficient retesting and modification of sets of neurostimulation parameters that were previously deemed to be of interest by the programming person. A set of neurostimulation parameters may have been deemed to be of interest because it successfully (or at least partially) treated pain at a targeted location, corresponds to a threshold of interest (e.g., a perception threshold or a muscle stimulation threshold), because it caused a certain response (e.g., muscle twitching) at a targeted location that can be used to define one or more maximum pulse parameters, and/or the like.

As indicated at step 518, one or more additional sets of neurostimulation parameters for which information was saved can be retested. This allows for efficient retesting of previously tested sets that were considered to be of interest. This also allows for efficient and effective comparisons of different sets, e.g., by retesting different sets (for which information was saved) one after the other, in a similar fashion as comparing different optical prescriptions during an optical exam when visiting an eye doctor.

In other embodiments, a user may be able to scroll through sets of neurostimulation parameters (for which information was saved for later retrieval) to have one or more of the sets retested without first displaying a list of such sets. For example, the name, rating and/or description of only a single set of neurostimulation parameters (for which information was saved for later retrieval) may be displayed at one time, during which time a user has an option to have that set retested (e.g., by pressing a predetermined button) or to progress to the next set (e.g., by pressing another predetermined button). For another example, as a user scrolls through sets of neurostimulation parameters (for which information was saved for later retrieval), the sets may be retested one after the other while the name, rating and/or description of the set currently being retested is displayed. Other variations are also possible.

In accordance with an embodiment, while a selected set of neurostimulation parameters is being retested, the programmer can accept, from a user, one or more adjustments to one or more of the pulse parameters of the set selected from the displayed list (i.e., the set being retested). In response to such adjustments being accepted from a user, the programmer will send instructions to the NS to make corresponding adjustments to the set being retested. This allows the user to fine-tune sets of neurostimulation parameters, as desired, to attempt to improve and preferably optimize therapy. While an adjusted set is being tested, the user can select a predetermined button (e.g., a “save”, “program” or “log” button) on the programmer if they decide that the set of neurostimulation parameters being tested (as adjusted) is of sufficient interest that information corresponding to the adjusted set should be saved. Depending on implementation, such information can overwrite the information corresponding to the set just adjusted, or a new information entry can be stored. It is also possible that one button (e.g., an “overwrite” button) would cause the information corresponding to the adjusted set to overwrite the information corresponding to the set just adjusted, and another button (e.g., the “program” or “log” button) would cause a new information entry to be stored. In other words, a snapshot created by adjusting a previous snapshot can overwrite the previous snapshot, or an additional snapshot can be saved.

As mentioned above when discussing step 506, the programmer can accept a rating from a user when the programmer accepts an indication from the user that the set of neurostimulation parameters being tested is of sufficient interest that information corresponding to the set of neurostimulation parameters being tested should be saved. In one embodiment, the rating can depend on how many times the user presses the predetermined button (e.g., the “program”, “log” or “save” button) that indicates that information corresponding to the set should be saved. For example, the user can have the option of pressing the button anywhere between one and N times (e.g., N equals 4), where how often the button is pressed indicates the rating. Alternatively, there can be one or more dedicated rating button(s). If there is a single rating button, the user can press the rating button anywhere between one and N times (e.g., N equals 4), where how often the rating button is pressed indicates the rating. There can alternatively be two ratings buttons (e.g., a green and a red button, or a plus and a minus button), one of which increases a rating when pressed, the other one of which decreases a rating when pressed. If there are N ratings buttons, the user can indicate a rating by pressing one of the N buttons. In still other embodiments, a drop-down or pop-up menu or a list box displayed in the programmer can be used to select a rating. Other variations are also possible. In some embodiments, ratings can range from 0 to N, where N is a positive value indicative of a maximum rating. In other embodiments, negative ratings can also be assigned.

In accordance with an embodiment, the information saved at multiple instances of step 508 can be binned based on how many times the predetermined button (which causes the saving) is selected by the user to indicate that information corresponding to the set of neurostimulation parameters being tested should be saved. More generally, the saved information corresponding to the sets of neurostimulation parameters can be binned based on the ratings associated with the stored information corresponding to the sets, thereby resulting in sets of neurostimulation parameters assigned the same rating being binned together.

Referring back to step 512, in accordance with an embodiment, the displayed list can be ordered in dependence on the binning, or more generally, in dependence on the ratings. For example, sets of neurostimulation parameters having the same rating can be binned together in a list (e.g., be listed next to one another in the list), or sets of neurostimulation parameters corresponding to a same lead and having the same rating can be binned together in a list. Additionally, sets can be ordered from highest to lowest ratings, or vice versa, in a displayed list.

In accordance with an embodiment, the programmer can be used to group together two or more of the tested sets of neurostimulation parameters for which the information was saved. In other words, one or more groups can be generated in response to a user, via the programmer, grouping together two or more of the tested sets of neurostimulation parameters for which the information was saved. Such groups can be generated, e.g., using the Group workspace shown in FIG. 4B. The programmer can also accept a name, from the user, for each group. Such names can be accepted as freeform text, and/or from one or more drop-down or pop-up menus or list boxes. In FIG. 4B, there are four groups with names: “Wake”, “Sleep”, “Sport” and “Sitting”. After groups are generated, the programmer can display a list of the one or more groups, e.g., by displaying the names assigned to the groups. In the “Group” workspace shown in FIG. 4B, the names of Groups are indicated on the tabs 456. In other words, the named tabs 456 can comprise the list of groups. In accordance with an embodiment, the programmer can accept, from a user, a selection of one of the one or more displayed groups (e.g., selection of one of the tabs 456), in response to which the programmer sends instructions to the NS that causes neurostimulation to be delivered in accordance with all of the sets of neurostimulation parameters included within the selected group (e.g., simultaneously, substantially simultaneously, consecutively, or on a scheduled basis, but not limited thereto). In an alternative embodiment, one of the groups can be selected when using one of the other workspaces, such as, but not limited to, the “Stim” workspace.

In accordance with an embodiment, a same set of neurostimulation parameters can be included in more than one group. For example, a set of neurostimulation parameters named “Neck Comforting” may be included in a group named “Wake”, as well as in a group named “Sleep”. In accordance with an embodiment, when the programmer accepts an adjustment to one or more pulse parameters associated with one of the sets of neurostimulation parameters (e.g., to the set named “Neck Comforting”), the saved information for that set can be modified such that whenever that set is again selected, or one of the groups that includes that set is again selected, neurostimulation is delivered in accordance with the adjusted one or more pulse parameters. In other words, wherever there is a change to a set of neurostimulation parameters that is in more than one group, the change globally affects every group within which that set is included. Accordingly, updating one set of neurostimulation parameters (i.e., one “lead set”) can globally update multiple groups (i.e., multiple “stimulation sets”).

A programming session during which numerous sets of neurostimulation parameters were tested can result in information corresponding to a reasonable number (e.g., up to twelve) of the tested sets being stored within memory of the NS and/or within memory of the clinical programmer. The maximum number of tested sets for which information can be stored may be the same for the NS and the programmer, or different (e.g., the programmer may be able to stored information corresponding to more sets, since the programmer likely contains a greater memory capacity).

At least some of the sets of neurostimulation parameters stored within the NS can be selected from for chronic treatment of pain. In one embodiment, all of the sets of neurostimulation parameters for which information is stored in the NS are available for selection by a patient using a patient programmer (e.g., 126). Alternatively, the programming person can specify, via the clinical programmer, which of the sets of neurostimulation parameters for which information is stored in the NS can be selected among by a patient using a patient programmer (e.g., 126). For example, referring back to the screenshot 454 in FIG. 4B, the programming person can use the “For Patient Use” button 464 to specify whether or not a patient can select specific sets of neurostimulation parameters and/or groups of such sets. This enables information for sets of neurostimulation parameters that are not useful for treating pain, but may be useful for defining maximum pulse parameters, or the like, to be stored within the NS and available for later retrieval by a programming person using a clinical programmer.

Information corresponding to sets of neurostimulation parameters that can be selected among by a patient using a patient programmer can be loaded into memory (e.g., 159) of a patient programmer (e.g., 126) in various different manners. In one embodiment, such information can be transferred from the clinical programmer to the patient programmer using the I/O circuitry (e.g., 146 and 156) of the two programmers. Alternatively, such information can be uploaded from the NS to the patient programmer, e.g., in response to the patient programmer being turned from off to on, or in response to a predetermined button (e.g., a load or connect button) on the patient programmer being selected. Information about how the various sets of neurostimulation parameters are grouped can also be loaded onto the patient programmer, to thereby enable a patient to select amount the various groups. It is noted that the NS can also store in its memory an “active set” and/or “active group” parameter, which specifies which set of neurostimulation parameters or group is currently activated/selected.

In the embodiments described above, the electronic circuitry (e.g., 118) of the NS and the components of the clinical programmer (e.g., 122) can be considered a data processing system of a neurostimulation system. Generally, the data processing system included in embodiments of the invention may include at least one processor (or other controller), which will typically include circuitry implanted in the patient, circuitry external of the patient, or both. When external processor circuitry is included in the data processing system, it may include one or more proprietary processor boards, and/or may make use of a general purpose desktop computer, notebook computer, handheld computer, or the like. The external processor may communicate with a number of peripheral devices (and/or other processors) via a bus subsystem, and these peripheral devices may include a data and/or programming storage subsystem or memory. The peripheral devices may also include one or more user interface input devices, user interface output devices, and a network interface subsystem to provide an interface with other processing systems and networks such as the Internet, an intranet, an Ethernet™, and/or the like. Implanted circuitry of the processor system may have some or all of the constituent components described above for external circuitry, with peripheral devices that provide user input, user output, and networking generally employing wireless communication capabilities, although hard-wired embodiments or other trans-cutaneous telemetry techniques could also be employed.

An external or implanted memory of the processor system can be used to store, in a tangible storage media, machine readable instructions or programming in the form of a computer executable code embodying one or more of the methods described herein. The memory may also similarly store data for implementing one or more of these methods. The memory may, for example, include a random access memory (RAM) for storage of instructions and data during program execution, and/or a read only memory (ROM) in which fixed instructions are stored. Persistent (non-volatile) storage may be provided, and/or the memory may include a hard disk drive, a compact digital read only memory (CD-ROM) drive, an optical drive, DVD, CD-R, CD-RW, solid-state removable memory, and/or other fixed or removable media cartridges or disks. Some or all of the stored programming code may be altered after implantation and/or initial use of the device to alter functionality of the stimulator system.

Embodiments of the present invention have been described above with the aid of functional building blocks illustrating the performance of specified functions and relationships thereof. The boundaries of these functional building blocks have often been defined herein for the convenience of the description. Alternate boundaries can be defined so long as the specified functions and relationships thereof are appropriately performed. Any such alternate boundaries are thus within the scope and spirit of the claimed invention.

Although the foregoing invention has been described in some detail by way of illustration and example, for purposes of clarity and understanding, it will be obvious that various alternatives, modifications and equivalents may be used and the above description should not be taken as limiting in scope of the invention

What is claimed is:

1. A method for use with a system including a neurostimulator, a programmer configured to send instructions to the neurostimulator, and one or more leads connected to the neurostimulator, wherein each lead includes one or more electrodes,
wherein the method is for enabling efficient identification of one or more preferred sets of neurostimulation parameters from among numerous possible sets of neurostimulation parameters, wherein each set of neurostimulation parameters specifies a lead, an electrode configuration for the specified lead, and one or more pulse parameters,
the method, comprising:
(a) sending instructions to the neurostimulator to cause testing of each of a plurality of different sets of neurostimulation parameters;
(b) saving, within the programmer and/or neurostimulator, information corresponding to a sub-plurality of sets of neurostimulation parameters,
wherein the sub-plurality corresponds to those tested sets of neurostimulation parameters for which an indication is accepted from a user, via the programmer, that information is to be saved, and
wherein a number of sets of neurostimulation parameters in the sub-plurality of sets of neurostimulation parameters is less than a total number of sets of neurostimulation parameters for which testing is caused;
(c) displaying, via the programmer, a list indicative of at least some of the sub-plurality of sets of neurostimulation parameters;
(d) accepting from a user, via the programmer, a selection of one of the sub-plurality of sets of neurostimulation parameters from the displayed list; and
(e) in response to one of the sub-plurality of sets of neurostimulation parameters being selected from the displayed list, sending instructions to the neurostimulator to cause retesting of the set of neurostimulation parameters selected from the displayed list without requiring that a user specify, via the programmer, the lead, the electrode configuration and the one or more pulse parameters corresponding to the selected set.

2. The method of claim 1, further comprising:
(f) while the set of neurostimulation parameters selected from the displayed list is being retested, accepting from a user, via the programmer, one or more adjustments to one or more of the pulse parameters of the selected set.

3. The method of claim 2, further comprising:
(g) saving new information, or modifying previously saved information, corresponding to the set of neurostimulation parameters adjusted at step (f), in response to accepting an indication from a user, via the programmer, that information corresponding to the adjusted set of neurostimulation parameters is to be saved.

4. The method of claim 1, wherein:
step (b) includes accepting from a user, via the programmer, a name, rating and/or description of each set of neurostimulation parameters of the sub-plurality for which information is saved; and
step (c) includes displaying the list indicative of at least some of the sub-plurality of sets of neurostimulation parameters by displaying the corresponding names, ratings and/or descriptions accepted at step (b).

5. The method of claim 1, wherein:
step (b) includes accepting from a user, via the programmer, a rating of each set of neurostimulation parameters for which information is saved; and
step (c) includes ordering the list in dependence on the ratings accepted at step (b).

6. The method of claim 1, wherein the one or more pulse parameters, for each set of neurostimulation parameters, includes one or more of a pulse amplitude value, a pulse width value and a pulse frequency value.

7. The method of claim 1, wherein the saving information corresponding to the sub-plurality of sets of neurostimulation parameters at step (b) includes:
for each of the plurality of different sets of neurostimulation parameters for which instructions are sent to the neurostimulator to cause testing,
(b.1) determining whether or not an indication is received from a user that information is to be saved for the set being tested at the time;
(b.2) if the indication is received from the user, then saving information corresponding to the set being tested at the time; and
(b.3) if the indication is not received from the user, then not saving information corresponding to the set being tested at the time.

8. The method of claim 7, wherein:
accepting an indication from a user that information is to be saved comprises accepting, via the programmer, a user selection of a predetermined button.

9. The method of claim 8, wherein:
step (b) includes binning the saved information based on how many times the predetermined button is selected by a user to indicate that information corresponding to the set of neurostimulation parameters being tested is to be saved; and
step (c) includes ordering and displaying the list in dependence on results of the binning.

10. The method of claim 1, further comprising:
(f) generating one or more groups in response to a user, via the programmer, grouping together two or more of the sets of neurostimulation parameters for which corresponding information was saved;
(g) displaying, via the programmer, a list of the one or more groups;
(h) accepting from a user, via the programmer, a selection of one of the one or more displayed groups; and
(i) in response to one of the one or more displayed groups being selected, sending instructions to the neurostimulator to cause the neurostimulator to deliver neurostimulation in accordance with all of the sets of neurostimulation parameters included within the selected group.

11. The method of claim 1, further comprising:
(f) generating two or more groups in response to a user, via the programmer, grouping together sets of neurostimulation parameters for which corresponding information was saved;
(g) accepting from a user, via the programmer, an adjustment to one or more pulse parameters associated with one of the sets of neurostimulation parameters included in at least two of the groups generated at step (f); and
(h) saving new information, or modifying previously saved information, corresponding to the set of neurostimulation parameters adjusted at step (g), in response to accepting an indication from a user, via the programmer, that information corresponding to the adjusted set of neurostimulation parameters is to be saved;
wherein after step (h), each of the groups that had included the adjusted set saved at step (h) prior to its adjustment, includes the adjusted set as saved at step (h).

12. The method of claim 1, wherein:
step (a) comprises sending instructions to the neurostimulator to test the plurality of different sets of neurostimulation parameters in response to a user manually specifying, via the programmer, the lead, the electrode configuration and the one or more pulse parameters corresponding to each of the sets of neurostimulation parameters to be tested.

13. The method of claim 1, wherein:
step (a) comprises sending instructions to the neurostimulator to test the plurality of different sets of neurostimulation parameters in response to the programmer at least partially automatically selecting one or more of the lead, the electrode configuration and the one or more pulse parameters corresponding to each of the sets of neurostimulation parameters to be tested.

14. A method of claim 1, wherein the neurostimulator comprises one of an implantable neurostimulator (INS), a non-implantable neurostimulator, and an at least partially non-implantable neurostimulator.

15. The method of claim 1, further comprising:
(f) accepting from a user, via the programmer, an indication that at least one of the sets of neurostimulation parameters for which information is saved is not for patient use.

16. The method of claim 1, further comprising:
(f) accepting from a user, via the programmer, an indication that information corresponding to one of the sets of neurostimulation parameters, for which information was saved, is to be deleted.

17. A programmer configured to communicate with a neurostimulator to which one or more leads are connected, wherein each lead includes one or more electrodes, the programmer comprising:
a controller configured to generate or accept a plurality of different sets of neurostimulation parameters to be tested, wherein each set of neurostimulation parameters specifies a lead, an electrode configuration for the specified lead, and one or more pulse parameters;
telemetry circuitry configured to send instructions to the neurostimulator to cause testing of each of the plurality of different sets of neurostimulation parameters;
memory;
a display; and
a user interface configured to accept, from a user, indications that information corresponding to a sub-plurality of sets of neurostimulation parameters being tested are to be saved in the memory of the programmer and/or within memory of the neurostimulator,
wherein a number of sets of neurostimulation parameters in the sub-plurality of sets of neurostimulation parameters is less than a total number of sets of neurostimulation parameters for which testing is caused;
wherein the controller is also configured to cause a list, indicative of at least some of the sub-plurality of tested sets of neurostimulation parameters for which a user indicated information is to be saved, to be displayed on the display;
wherein the user interface is also configured to accept, from a user, a selection of one of the tested sets of neurostimulation parameters from the displayed list; and
wherein the controller is also configured to cause, in response one of the tested sets of neurostimulation parameters being selected from the displayed list, the telemetry circuitry to send instructions to the neurostimulator to cause retesting of the set of neurostimulation parameters selected from the displayed list without requiring that a user specify the lead, the electrode configuration and the one or more pulse parameters corresponding to the selected set.

18. The programmer of claim 17, wherein:
the display and the user interface collectively comprise a touch screen controlled by the controller.

19. The programmer of claim 18, wherein:
a screen displayed via the touch screen includes at least one predetermined button that, when selected by a user, provides the indication that information corresponding to the set of neurostimulation parameters being tested is to be saved in the memory of the programmer and/or within the memory of the neurostimulator.

20. A system comprising:
a neurostimulator including memory;
one or more leads connected to the neurostimulator, wherein each lead includes one or more electrodes;
a programmer including a controller, memory, telemetry circuitry, a display and a user interface;
wherein the controller of the programmer is configured to generate or accept a plurality of different sets of neurostimulation parameters to be tested, wherein each set of neurostimulation parameters specifies a lead, an electrode configuration for the specified lead, and one or more pulse parameters;
wherein the telemetry circuitry of the programmer is configured to send instructions to the neurostimulator to cause testing of each of the plurality of different sets of neurostimulation parameters;
wherein the user interface of the programmer is configured to accept, from a user, an indication that information corresponding to a set of neurostimulation parameters being tested is to be saved in the memory of the programmer and/or within the memory of the neurostimulator;
wherein the controller of the programmer is also configured to cause a list, indicative of a sub-plurality of the tested sets of neurostimulation parameters for which a user indicated information is to be saved, to be displayed on the display of the programmer,
wherein a number of sets of neurostimulation parameters in the sub-plurality of sets of neurostimulation parameters is less than a total number of sets of neurostimulation parameters for which testing is caused;
wherein the user interface of the programmer is also configured to accept, from a user, a selection of one of the tested sets of neurostimulation parameters from the displayed list; and
wherein the controller of the programmer is also configured to cause, in response one of the tested sets of neurostimulation parameters being selected from the displayed list, the telemetry circuitry of the programmer to send instructions to the neurostimulator to cause retesting of the set of neurostimulation parameters selected from the displayed list without requiring that a user specify the lead, the electrode configuration and the one or more pulse parameters corresponding to the selected set.

21. A method for use with a system including a neurostimulator, a programmer configured to send instructions to the neurostimulator, and one or more leads connected to the neurostimulator, wherein each lead includes one or more electrodes,
wherein the method is for enabling efficient identification of one or more preferred sets of neurostimulation parameters from among numerous possible sets of neurostimulation parameters, wherein each set of neurostimulation parameters specifies a lead, an electrode configuration for the specified lead, and one or more pulse parameters,
the method, comprising:
(a) effecting testing of a plurality of different sets of neurostimulation parameters;
(b) during the testing effected at step (a), saving, within the programmer and/or neurostimulator, information corresponding to a sub-plurality of sets of neurostimulation parameters being tested, in response to accepting indications from a user that the sub-plurality of sets of neurostimulation parameters being tested are of interest,
wherein a number of sets of neurostimulation parameters in the sub-plurality of sets of neurostimulation parameters is less than a total number of sets of neurostimulation parameters for which testing is effected at step (a);
(c) accepting, from a user, a selection of a previously tested set of neurostimulation parameters for which information was saved at step (b); and
(d) in response one of the previously tested sets of neurostimulation parameters being selected at step (c), effecting retesting of the set of neurostimulation parameters selected at step (c) without requiring that a user specify the lead, the electrode configuration and the one or more pulse parameters corresponding to the selected set.

22. A non-transitory computer readable medium, including instructions stored thereon which when read and executed by one or more processors cause the one or more processors to perform the steps comprising:
(a) sending instructions to a neurostimulator to cause testing of each of a plurality of different sets of neurostimulation parameters;
(b) saving information corresponding to a sub-plurality of sets of neurostimulation parameters,
wherein the sub-plurality corresponds to those tested sets of neurostimulation parameters for which an indication is accepted that information is to be saved, and
wherein a number of sets of neurostimulation parameters in the sub-plurality of sets of neurostimulation parameters is less than a total number of sets of neurostimulation parameters for which testing is caused;
(c) displaying a list indicative of at least some of the sub-plurality of sets of neurostimulation parameters;
(d) accepting a selection of one of the sub-plurality of sets of neurostimulation parameters from the displayed list; and
(e) in response to one of the sub-plurality of sets of neurostimulation parameters being selected from the displayed list, sending instructions to the neurostimulator to cause retesting of the set of neurostimulation parameters selected from the displayed list without requiring that a user specify, via the programmer, the lead, the electrode configuration and the one or more pulse parameters corresponding to the selected set.
